(19) United States
(12) Patent Application Publication
(10) Pub. No.: US 2004/0064065 A1
(43) Pub. Date: Apr. 1, 2004

(54) Individual illusion system

(76) Inventor: John Anthony Pescatore JR., Honolulu, HI (US)

Correspondence Address: John A. Pescatore, JR., 2345 Ala Wai Blvd. #1702, Honolulu, HI, 96815, US

(21) Appl. No.: 10/669,674

(22) Filed: Sep. 23, 2003

Related U.S. Application Data
(60) Provisional application No. 60/412,939, filed on Sep. 24, 2002.

(51) Int. Cl. A61B 13/00

(52) U.S. Cl. 600/558

(57) ABSTRACT

The optical layout of the invention is designed to asymmetrically stimulate at least the magnocellular and parvocellular pathways of the two eyes during binocular fusion. Furthermore, the optical layout of the invention may be adjusted to adapt to individual visual processing needs. The present invention describes a system of optics useful in furthering studies of the primate visual processing system and finding clinical applications. For the majority of the population applications of the optical system could serve instructional purposes as well as a form of entertainment. The elicited illusions are dependent on the specific individual and therefore the system distinctly allows for customization.

### CROSS-REFERENCE TO RELATED APPLICATIONS

Not Applicable

### STATEMENT REGARDING FEDERALLY SPONSORED RESEARCH AND DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of Invention

This invention relates to the preparation of ambient lighting and of light reflected or emitted from a display in order to incite particular neural processes of the primate visual processing system. More specifically the ratios of visual stimuli under mesopic vision to at least the parvocellular and magnocellular systems of each of the two eyes are made independently adjustable prior to binocular fusion. Application of the invention to the binocular fusion of viewed images, particularly moving images, is of a universal nature because it is not restricted to any particular display technology or data storage technology and is fundamentally accomplished with passive optics.

2. Description of the Related Art

Understanding the complexity of human vision, like most of human physiology, is an ongoing study that is at least centuries old. The eyes may be considered as windows into the brain: light transmitted through the eyes affects cellular organizations within the retina. The retina is brain tissue with an architecture that may simply be described as a stack of layers comprised of different cell types and their intercellular neurological connections. An excellent account of the progress of the study of vision (complete with references to the original scientific literature) from the neuroanatomy of the retina, as highlighted with video graphics, through experiments in psychophysics can be found at http://webvision.med.utah.edu. This electronic tutorial fundamentally divides the visual process into vertical pathways, from the eye to higher processing centers within the deeper recesses of the brain, and lateral pathways that network cellular organizations within the retina. The description of the pathways of the˜million nerve fibers bundled into each human optic nerve is detailed to a midpoint of the brain and then superficially described onto higher visual processing centers of the brain. An overview of the neurobiology of vision by Livingstone, M. S., et. al., “Segregation of Form, Color, Movement, and Depth: Anatomy, Physiology, and Perception,” Science, 240: 740-749 (1988) is representative of that of neurobiologists who study the higher visual processing centers of primates' brains.

Basically all cells, as those of the retina, have an associated electromotive force across their membranes. This force is maintained by concentration gradients of ions and/or specific molecules across semi-permeable cell membranes. These gradients are dynamic with a constant flux of such species. The initiation of visual transduction can be thought as: photoisomerizations of chromophores whose resultant conformational changes within primary photoreceptor cells induce a change in the permeability of the primary photoreceptor cell's membrane. (Furthermore, transduction is complicated with enzymatic reactions that regulate the process, as well as specific molecules called neurotransmitters that must bind to correspondingly specific receptor sites). With the thousands of chromophore molecules contained in any one such photoreceptor cell, it is understood that the resulting changes to the permeability of the cell membrane upon photoisomerizations will grossly affect concentration gradients across the membrane. These resultant changes in the cascade of neurotransmissions affect in turn the concentration gradients of neighboring cells, not necessarily being other primary photoreceptor cells. The types of species (i.e., cell types, neurotransmitters and ions) involved in intercellular contacts, as well as the changes of electromotive forces, are detailed in the literature. In brief there are networks of local circuits consisting of neighboring cell types that may act synergistically or antagonistically to start characterizing the light (e.g., wavelength, intensity) impinging across the surface of the retina. Two regions of the complex architecture of our visual process are discussed in detail: the primary photoreceptor cells of the retina and the cells of thalamus, which are relevant to the invention.

Microscopically resolved primary photoreceptor cells are either cone shaped or rod shaped. Both cell morphologies are loaded with proteins containing retinal (polyene) chromophores: these polyene molecules may absorb photons of certain energies within the range of wavelengths comprising visible light. Upon absorption of a photon, the chromophore molecule undergoes an electronic energy state transition. (A molecular electronic energy state may be considered as a specific configuration of all of a molecule's electrons). The transition is from the lowest energy (ground) electronic state of the chromophore to one of its excited electronic states. In an excited electronic state, the polyene molecule isomerizes, which triggers a complicated cascade of neural transmissions, eventually along the optic nerve. There are three classes of cone shaped cells; the classes are defined by the lambda maxima of their inhomogeneously broadened optical absorption profiles, which are built upon these electronic energy state transitions. The inhomogeneously broadened optical absorption profile of the polyene chromophores in the rods has a lambda maximum at 498 nm. The contrasts of the molecular environments of the different proteins containing the polyene chromophores of the rods and of the cones are responsible for the differences of the inhomogeneously broadened optical absorption profiles. All four human proteins (of the rods and of the three classes of cones) have their amino acids sequenced, thus their primary structures are determined; as well as, secondary, tertiary, and quarternary structures modeled based on studies of bovine rhodopsin, as discussed by Nathans, Jeremy, et.al., “Molecular Genetics of Human Color Vision: The Genes Encoding Blue, Green, and Red Pigments,” Science, 232: 193-202 (1986) and by Okada, Tetsuji, et.al., “Functional Role of Internal Water Molecules in Rhodopsin Revealed by X-Ray Crystallography,” Proceedings of the National Academy of Sciences USA, 99(9): 5982-5987 (2002).

Although the distribution of rods and cones is not symmetric throughout the retina, the distribution is not random. Generally speaking the distribution of cones is concentrated in the center of the visual axis called the fovea and diminishes irregularly toward the periphery of the retina. Our center of gaze is defined by a 0.04 mm² area of our retina that is comprised solely of cones. More specifically this region called the foveal pit, devoid of rods, is comprised of the two most abundant classes of cones, which have the two lower electronic energy state transition maxima (i.e., these optical absorption lambda maxima are 558 nm and 531 nm versus 420 nm for the three cone classes; colloquially referred to as red, green and blue, respectively). The third class of cone shaped primary photoreceptor cells, with the highest electronic energy state transition maximum, is randomly distributed outside the foveal pit and comprises less than 10% of the total cone cell population. Rod cells comprise the bulk of the primary photoreceptors outside the fovea and outnumber the sum of the three classes of cone cells throughout the retina by a cell count ratio of roughly 30:1, as discussed by Curcio, C. A., et.al., “Distribution of Cones in Human and Monkey Retina: Individual Variability and Radial Asymmetry,” Science, 236: 579-582 (1987) and by Mollon, J. D., et.al., “The Spatial Arrangement of Cones in the Primate Fovea,” Nature, 360: 677-679 (1992).

During daylight or photopic conditions the effects of irradiated cones are more pronounced than rods and allow us high visual acuity as well as color vision. Note that it is the goal of commercial lighting to stimulate photopic vision. Likewise the display industry is focused on the production of displays that are useful under photopic vision. In other words commercial lighting displays and general illumination must target the cones to utilize our visual acuity and our perception of color. As ambient illumination decreases the rods contribute more in affecting higher order vision processes; this stage of visual processing is mesopic vision. Characteristics include seeing less clearly and a decrease in color saturation. An example is outside night vision under dim artificial lighting. Under the dimmest broadband illumination conditions, humans are not aware of color and cannot clearly resolve objects and are using scotopic vision, essentially the effects of rods only. For example is our sense of vision while walking at night in a field illuminated by the sun's reflection from the moon. Scotopic vision results in foveal blindness or the inability to focus sharply since foveal cones are not stimulated. Nevertheless scotopic vision quickly becomes mesopic with the introduction of color. For example, the albeit dim artificial illumination of colored emission from a light emitting diode would recruit cones into our night vision.

Visual acuity is based on the sizes of the photoreceptor cells and their intercellular interactions. The sizes of the cones are smaller in the fovea than in the periphery of the retina. Furthermore, within the fovea the cells comprising the layers above the primary photoreceptor cells are displaced concentrically outward to decrease scattering of any light incident on the fovea. These cellular displacements actually define the foveal pit. The limit of visual resolution of the unaided eye is a function of the area of such an underlying single foveal cone cell. Ganglion cells connect the network of cells of the retina to those of the thalamus through the optic nerve. In humans the more common ganglion cell types are named the parasol and the midget. The smallest sized cones of the foveal pit are networked throughout the retina with analogously reduced size cells and together comprise a midget ganglion cell pathway to higher processing centers of the brain. The midget ganglion cell pathway is therefore specialized for high visual acuity or resolution. The midget ganglion cell pathway has nearly one to one neural connections to the smallest sized cones. At the other extreme are estimates of 75,000 rods networking through several thousand secondary photoreceptor cells onto a larger parasol ganglion cell. These parasol ganglion cells define a ganglion cell pathway other than the midget that fundamentally links the peripheral retina to the thalamus. In this sense, the networking of the majority of cones outside of the fovea can be likened to that of the rod network; these perifoveal cones are not connected to the midget ganglion cell pathway. The diffuse nature of the intercellular network of the retina periphery is associated with lower visual acuity. This example of extremes in intercellular connectivity is exemplary of a more general bifurcation of our visual neurology. The midget ganglion cell pathway terminates in specific layers comprised of small cells of the lateral geniculate nucleus of the thalamus, in contrast to the layers of the lateral geniculate nucleus containing larger cells.

The composition of the lateral geniculate nucleus of the thalamus may be described as four parvocellular layers on top of two magnocellular layers. The parvocellular layers, developed in select primates, are associated with brain centers for perceiving color and acuity. The number of geniculate cells comprising the dorsal four parvocellular layers is about ten times greater than that of the two ventral magnocellular layers. The magnocelluar layers contain larger geniculate cells (thus, thicker axons) and are associated with quicker propagation of nerve impulses. The magnocellular layers are evolutionarily older and are neurally connected to areas of the brain responsible for perceptions of e.g., depth, space, luminosity and motion. A fraction of the ˜million ganglion cells within a human optic nerve defines a midget ganglion cell pathway from foveal cones to the parvocellular layers. Another fraction of the million ganglion cells within an optic nerve connects perifoveal cones and rods by a parasol ganglion cell pathway to the magnocellular layers. After the right and left optic nerves meet at the thalamus they essentially are multiplexed and routed onto the various processing centers of the brain.

There are works that associate irregularities in the magnocellular layers with dyslexia, such as Livingstone, M. S., et.al., “Physiological and Anatomical Evidence for a Magnocellular Defect in Developmental Dyslexia,” Proceedings of the National Academy of Sciences USA, 88, 7943-7947 (1991). There are a number of patented processes that serve to specifically visually stimulate the magnocellular pathways for either diagnostic (U.S. Pat. No. 5,474,081), therapeutic (U.S. Pat. No. 6,443,572) or both purposes (U.S. Pat No. 6,213,956) in the study of dyslexia. Our invention thus may find such clinical applications.

The study of how humans perceive visual stimuli, based on physical measurements as well as intangible individual interpretations, is a subject of psychophysics. Optical illusions may be considered as an integral part of psychophysics. The optical illusions existent when viewing with one eye as opposed to those requiring both eyes can be distinguished. Furthermore optical illusions may be the result of active optics or of passive optics; that is, whether or not electrical power is required to create the illusions.

Stereopsis is the ability to gauge depth based on the perceived differences of light upon comparison between the two eyes. The primate eye is a compound lens with the largest refraction of light occurring at the interface of the cornea with air. An adjustable lens called the crystalline lens is found behind the pupil. The resting primate eye is focused at infinity and the muscular adjustments made on the shape of the crystalline lens to further refract a specific image to a focus onto the retina is called accommodation. In order to compare the left and right eyes' accommodations each whole eye moves, this process called convergence, until binocular fusion into a single perceived image. Convergence assumes that a single object is focused upon to acquire a binocular fusion. If for example two laterally separated images are to be viewed independently but simultaneously, one by each eye, to induce binocular fusion then the process is distinguished as divergent fusion. Although accommodation and convergence may quickly yield a single sharply focused view, one can easily disrupt the process of stereopsis with unnatural stimuli—such as evident with divergent fusion.

For example if one eye views a white circle on a black square and the other eye views identical sizes and shapes but opposite contrast—a black circle on a white square, then after binocular fusion into one perceived image (i.e., as a result of divergent fusion) the luminance contrast will oscillate. This shifting of luminance contrasts can also be induced by color contrasts and is termed retinal rivalry. Retinal rivalry can be considered an extreme disturbance to stereopsis inasmuch as more conspicuous disparities would preclude binocular fusion. Three dimensional optical illusions can be obtained by binocular fusion of slightly differing two dimensional images; analogous to the differences of light perceived by each eye during stereopsis when viewing a three dimensional object.

Historically, Wheatstone, discussed by Livingstone, M. S., et.al., “Psychophysical Evidence for Separate Channels for the Perception of Form, Color, Movement, and Depth,” Journal of Neuroscience, 7(11), 3416-3468 (1987), is credited for identifying that divergent fusion of two slightly different images results in a perception of depth. Further developments led to anaglyphs, which are contrasts in color between two images. Usage of appropriately colored filters allows each eye to perceive only one of the two images. A convenience of overlaying the two images is allowed if the anaglyphs are produced on transparent substrates. Such images that differ enough to allow binocular fusion and induce a sense of depth are called stereograms. Stereograms are now fundamentally produced by photographing a scene from two different angles reminiscent of the viewpoints of the two eyes. Each point of view may be divergently fused for perception of depth. If the images are on transparent substrates, such as film, two projections may be superimposed. To induce depth perception, the projected images must be viewed independently and simultaneously by each eye, as restricted with the anaglyph method or by the usage of orthogonal polarizations. In general such concepts are referred to as stereoscopic or three dimensional, 3D. Note that the stereoscopic effects are not necessarily restricted to stationary images.

An early 1900 report by Pulfrich, discussed by Livingstone, M. S., et.al., “Psychophysical Evidence for Separate Channels for the Perception of Form, Color, Movement, and Depth,” Journal of Neuroscience, 7(11), 3416-3468 (1987), identified a moving stereoscopic effect such as found in the observation of the lateral oscillation of a swinging pendulum with one eye covered by a neutral density filter. The perceived trajectory of the pendulum bob is not in a vertical plane but in a tilted plane, tracing out an ellipse. An explanation of this perceived elliptical orbit is based on the different visual processing times of the two eyes and a correlation between the distance of the moving pendulum bob to the optically attenuated eye. The Pulfrich effect is incorporated in clinical optical evaluations as described in several patents, e.g., U.S. Pat. No. 5,347,330. The classical usage of passive optics for utilizing the Pulfrich effect in creating a 3D television image has been described in U.S. Pat. No. 3,445,153. The U.S. Pat. No. 3,445,153 teaches that inducing a Purkinje shift by optical attenuation of only one eye results in the Pulfrich effect when viewing moving images. The Purkinje shift is the shift between the absorbances of the primary photoreceptors: between the rods and the cones. Improvements of this application as well as incorporating spectral contrasts (such as an anaglyph methodology) to enhance stereoscopic viewing are described in U.S. Pat. Nos. 4,131,342, 4,836,647 and 6,144,440.

The approach to creating stereoscopic images especially moving images based on the presentation of two images differing in resolution has been disclosed in U.S. Pat. No. 5,748,382 and similarly in U.S. Pat. No. 6,252,982. In contrast to these works the present invention does not require active optics. In general, modem three dimensional television images are based on applications of advanced optoelectronic technologies, e.g., U.S. Pat. Nos. 6,304,263, 6,122,000, and 6,078,423. Ultimately, the general population of viewers of 3D movies awaits the presence of affordable full color holographic video for entertainment. In brief modem stereoscopic moving images achieved in 3D television may be divided into two groups. Both groups require active optics to alternately present images to be fused in the brain. One stereoscopic approach is to alternately modulate the transmission into each eye with goggles containing active optics. The other group does not require goggles, thus distinguished as autostereoscopic, but instead modulates the transmission of two viewpoints at the display. Because there are neither added active components nor massive electronic computations, the present invention is not considered as belonging to the mature field of three dimensional television. Instead the present invention is an application of classical optics made suitable for inducing illusions that are better realized with improvements in display technology. In this regard, any incandescent or luminescent display is adequate, where advancements made in pixel resolution and display brightness will necessarily improve the illusions. In this regard, any color display of past, present, or future technology is appropriate to realize the principles of the invention. Our invention stimulates the cells of the retina in monocular fashion so that binocular fusion at the thalamus of the information from the optic nerves is of an asymmetric nature. Keigo Iizuka describes in a 2003 article in Review of Scientific Instruments, volume 74(8), pages 3636-3639, an elegant application of cellophane to orient the polarization of the light from any type of linearly polarized light emitting display in order to be used for stereoscopic viewing. This application is strictly to distinguish images based on polarization.

It is considered that the Pulfrich effect is resultant of asymmetrical stimulation of the magnocellular and parvocellular pathways with respect to each of a viewer's two eyes. The asymmetry is that one eye sees for example a sharp pendulum bob, stimulating parvocellular paths, while the other eye sees motion of low luminosity contrasts, stimulating magnocellular paths. Since the perceived trajectory of the exemplary pendulum is not accurate and the optically attenuated eye feels “patched” upon prolonged viewing, the Pulfrich effect is not desirable for viewing movies. Likewise, the anaglyph method is not favorable because of the inability to reproduce a full spectrum of colors. Based on these lessons, there is a need for control of asymmetrical magnocellular and parvocellular stimuli in order to achieve a sense of balanced light intensities to the viewer as well as a full spectrum of colors.

### BRIEF SUMMARY OF THE INVENTION

Illusions of depth and effects of color opponency are manifested in the present invention by asymmetrically preparing light prior to presentation to the two eyes of a viewer with normal efficiency in terms of visual processing capabilities. The illusions are so defined in context to entertainment, but the system of optics may also find usage in scientific as well as pedagogical endeavors. The resultant illusions are presumed to be based on the discrepant stimuli initiated in each eye to propagate along the neural parvocellular and magnocellular pathways. The discrepancies are fundamentally made in size, shape, color, hue, luminosity contrast, resolution, and brightness between images viewed with mesopic vision. It is not unlikely that a viewer will describe these innate responses as hallucinations.

The optical layout of the invention is designed to affect the primary photoreceptors as well as the cells of the thalamus to asymmetrically stimulate the magnocellular and parvocellular pathways in an unnatural manner, which may be adjusted to adapt to individual visual processing needs. The present invention is further drawn to an asymmetric preparation of light for binocular fusion such that the viewer does not perceive a strong sense of imbalance of light intensities. The present invention describes a system of optics useful in furthering vision studies and may find further clinical applications. For the majority of the population applications of the optical system could serve instructional purposes as well as a form of entertainment. The elicited illusions are dependent on the specific individual and therefore the system distinctly allows for customization.

In order to incite asymmetrical optical responses from a viewer, thus optical illusions, the present invention relies on three principle actions. First, the invention calls for recruitment of the rod cells of the retinal periphery into the visual processes as obtainable with control of ambient lighting conditions. Dimming room lights and thus background illumination, as well as the usage of especially designed eyewear, helmets, and/or opaque barriers would quickly dilate the pupils and bring the rod cells into the process. More importantly, in addition to the anatomical reflexes of the iris in dilating the pupil, dim illumination results in mesopic vision at the biochemical level of enzymatic interactions. Second, the amount of light absorbed by the foveal cones of one of the viewer's eyes is attenuated to a fraction of the absorption by the remaining eye's cone cells. Third, the images to be viewed are made adjustable in characteristics that further stimulate and thus distinguish the neural pathways of the primary photoreceptors selected by optical and/or spatial filtration. In this manner the imbalance of the absorption of light between the two eyes will cause an asymmetrical optical response, which results in illusions dependent on an individual basis.

The fundamental asymmetrical optical response is likened to the Pulfrich effect. The invention furthers the Pulfrich effect by using mesopic vision as a starting point. In this sense the Purkinje shift between the absorbances of the primary photoreceptor cells becomes more or less a tunable shift. The shift induced by the invention is not restricted to that between rod or cone based vision but a shift that changes the ratios of the contributions of the types of primary photoreceptor cells. The invention then further stimulates the fundamental pathways of neural transmission of the favored primary photoreceptor cells as selected by the Purkinje shift. These invented stimuli are adjusted to resonate with the distinguishing features of the neural pathways associated with the selected primary photoreceptor cells. The invented stimuli may be created with passive optics and amplify the asymmetry of the innate optical responses between each eye. Such stimuli are selected such that the magnocellular or parvocellular systems are favored and include but are not limited to size, shape, color, hue, luminosity contrast, resolution, and brightness of the images.

In the simplest embodiment of the invention a color stationary image, e.g., a photograph is viewed under mesopic conditions. Under such mesopic conditions, where both rods and cones are active, one of the viewer's eyes is filtered of wavelengths longer than ˜500 nm. This attenuates the absorption of light by the foveal cones as well as perifoveal cones of the same two classes, “red” and “green”. Retinal rivalry based on color contrasts can be avoided by adjustment of the degree of optical attenuation. Since rod cells outnumber cone cells, optically attenuating the transmission of light of wavelengths greater than 500 nm under mesopic vision will allow rods the dominant primary photoreceptor response. By viewing a color image under such conditions, one eye's retina may receive a full spectrum of colors. The other eye's image is not as colorful and is viewed with lower acuity since its image is filtered of visible light resonant with the foveal cone cells thus is based on light absorbed with rods and the perifoveal “blue” class of cone cells. Further adjustments to the intensity of light reaching each eye include usage of spatial filters, such as an aperture to limit the perifoveal irradiation through the dilated pupil, and/or a (broad spectrum) neutral density filter. Such adjustments, common to the art, are made to the intensity and wavelength distributions until a comfortable imbalance is perceived.

In another embodiment of the invention a pair of images such as photographs is viewed. The photographs should be identical or nearly so except that one is monochrome (e.g., black and white) and the other is color. In this embodiment of the invention the optical filters for blocking wavelengths greater than ˜500 nm are replaced by the presence of the additional monochrome image. A merit of this invention is the absence of separately photographed scenes. Instead one full color scene and a copy made nearly devoid of color are used to elicit an illusion of depth in an image with a full spectrum of colors. Simultaneously viewing two photographs is best done with a lens system, one system per eye, such as commonly employed in a conventional stereoscope. These embodiments of optical layouts for viewing stationary images are applicable for example in reading text, most likely under clinical settings.

A dual lens system, such as that of a microscope, may be utilized to simplify binocular fusion and is useful in other manners, common to the art. For example, instead of fixating on an object, images may be created that require the viewer to focus at infinity. This puts the viewer's eyes in a relaxed state and alleviates strain from prolonged viewing. Another useful aspect of dual lens optics is that angular magnification is possible. Thus instead of requiring a large display object, projection of a defined image onto the whole of the retina gives increases in magnification over a single lens system. The overall magnification is necessarily chosen by the viewer and is dependent on the resolution of the image to be viewed. A poorly resolved image, for example a grainy photograph, will appear mosaic under high enough magnification. The usage of two dual lens systems, one for each eye, allows for facile divergent fusion and magnification of two similar images.

The present invention relates to a method of viewing two dimensional objects (e.g., a display or an image) using an optical layout that induces illusions, such as of depth. Although the optical illusions are noticeable using stationary objects such as photographs, they are much more dramatic when the objects are either light emitting displays or their images. There are several orders of magnitude in lighting contrasts gained when utilizing for example a high resolution video display versus that of a high resolution photograph. Moreover light emitting displays can be reflected in a manner to further accentuate the luminosity contrasts within the reflections. The range of luminosity contrasts thus expanded gives further perceptions of depth. Perceptions of color also benefit since the image is not restricted in contrasts of hue. In other words, ranges of shades of color and of luminosity contrasts within an image become possible with light emitting displays that are not possible with images that are illuminated only by reflection (as are photographs).

The most preferred embodiment of the invention is when visual results are perceived when the optical layout is combined with the benefits added by motion as perceived in viewing video motion pictures. Relative motion and occlusion among the objects within a video scene, afforded either across the video screen or by the recording camera's change in focus upon objects within the field of view, stimulates the perception of the third dimension of depth. In general the invention is applicable to viewing 2D moving color images regardless of the display technology or the data storage technology. An object of the invention is to induce illusions while viewing any two dimensional color display technology. This object may be realized with almost trivial applications of passive optics. In terms of entertainment since only passive optics is required to induce illusions, the invention may be used as an accessory to essentially any display. A secondary object of the invention is to relieve the viewer of eyewear while fulfilling the primary object. Another object of the invention is to construct an optical layout useful in furthering the study of the biology of vision—potentially at the clinical level. A better understanding of the invention is made with reference to the following detailed descriptions and accompanying figures.

### BRIEF DESCRIPTION OF THE SEVERAL VIEWS OF THE DRAWING

FIG. 1A illustrates a first embodiment of the invention;

FIG. 1B illustrates a variation of the FIG. 1A embodiment;

FIG. 1C illustrates another variation of the FIG. 1A embodiment;

FIG. 2 illustrates a second embodiment of the invention;

FIG. 3 illustrates a variation of the FIG. 2 embodiment and illustrates a preferred embodiment of the invention;

FIG. 4 illustrates a third embodiment of the invention.

### DETAILED DESCRIPTION OF THE INVENTION

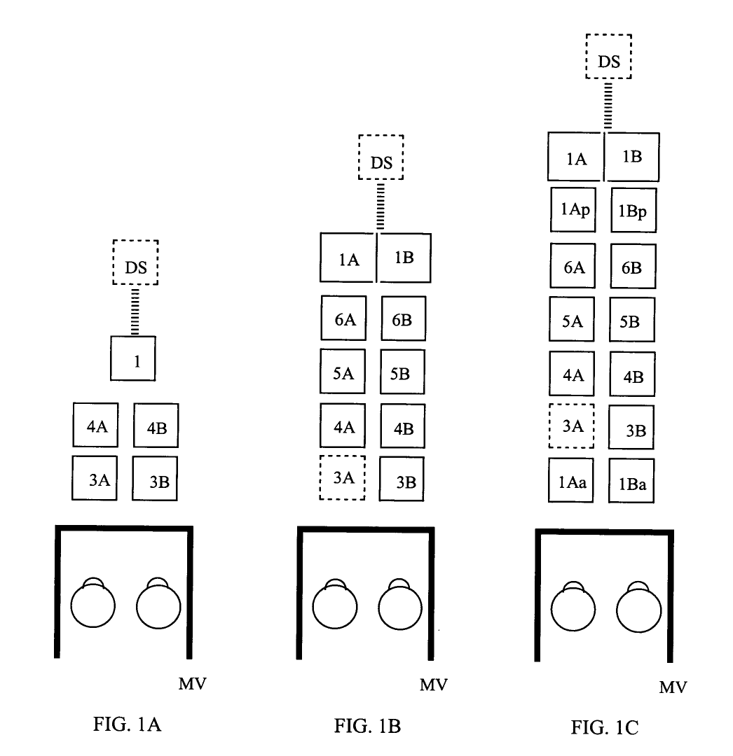

In the simplest embodiment of the invention a single color display with one image is viewed utilizing the optical layout depicted in FIG. 1A. Block 1 exemplifies the color image to be viewed (e.g., a color photograph, an electronic color display or a projected color image). If an image to be viewed is from the conversion of data from storage to display, then the representation of this process is the dotted box labeled DS. We emphasize that the invention does not have any limits on the form of data storage technology or its conversion to any display technology. In this regard the box DS can represent a live television feed, a computer graphics file or even a holographically stored image to be projected via tiny mirrors. Consequently, it is understood that in all embodiments of the invention there is a universal application regardless of the display technology or the data storage technology. Border MV represents a means of dimming the viewer's ambient lighting conditions as well as blocking peripheral vision (e.g., an especially designed helmet or a light enclosure that is not necessarily worn by the user of the invention) in order to obtain mesopic vision. Block 3A is a combination of optical filters that attenuates light of wavelengths resonant with the foveal cone cells, e.g., longer than ˜500 nm, leaving the light absorbed by the rod cells nearly unattenuated. Other examples of the representation of Block 3A may include: a combination of filters that selectively attenuates absorption by all cone classes including blue, a combination of filters that selectively attenuates absorption by mostly the red cone class, a combination of filters that selectively attenuates absorption by mostly the green cone class, or a combination of filters that selectively attenuates absorption by mostly the blue cone class and the red or green cone class. Block 3B is a spatial (opaque) filter in the form of an aperture that partially blocks the light falling upon the perifoveal retina.

In this fashion, Block 3A represents optical attenuation of light stimulating the parvocellular pathways and allows a more pronounced magnocellular response to the light impinging the eye behind Block 3A. On the other hand, Block 3B physically limits the amount of light onto the photoreceptors of the magnocellular pathways and favors a parvocellular response from the eye free of optical attenuation; the spatial filter Block 3B not being wavelength selective. The combination of filters is thus preferentially determined by an individual with the purpose of preparing light to asymmetrically stimulate the two fundamental neural processing pathways of each eye. The balance of light intensities falling upon each eye may be independently fine adjusted with broad spectrum optical attenuation filters. Blocks 4A and 4B represent these broad spectrum optical attenuation filters such as variable neutral density filters or pairs of plane (plastic sheet) polarizers. (The two sheet polarizers are selectively overlaid to transmit a fraction of the incident light intensity). The optics most relevant to realization of the invention are Border MV and Block 3A. In other words, to view Block 1 in fulfillment of the aspects of the invention Blocks 4 and 3B are considered useful yet not mandatory to creating an imbalance (asymmetric stimulation) of the parvocellular and magnocellular pathways. The stated examples are not exhaustive and the arrangement of the passive optics may be interchangeable with other possibilities construed by those of skill in the art. For example, the passive filter combinations may be employed in goggles while viewing projected stationary or motion picture images.

Computer generated graphics, or more generally user control of an electronics display, has several aspects useful to the invention. First, any aspect of an image may be controlled e.g., range of color, hue, luminosity contrast, etc. Second, the coordinate system of the display may be adjusted for physical manipulation of the graphics. Third, duplicate images may be generated on the same display (i.e., the same video graphics monitor). In this fashion user control of images may determine any number of combinations of lighting and/or geometric disparities between the two images in fulfillment of the scope of the invention. This variation of the embodiment shown in FIG. 1A is drawn in FIG. 1B. Block 1 is now divided into Blocks 1A and 1B to represent the two images within one display. The images represented by Blocks 1A and 1B are simultaneously viewed with one eye through the A series of optics and the other eye through the B series. The Block 3A would most likely not be required as its function could be handled by electronic control of the features of the image of Block 1A. Block 3A is drawn in a dotted frame to highlight this possibility. In order to facilitate divergent binocular fusion the two images are conventionally viewed through lens systems. Blocks 5A and 5B represent two lens systems (either single or dual) in FIG. 1B. Common to the art, even a single lens system may be comprised of multiple lenses in order to compensate for any of a number of aberrations, such as spherical or chromatic. Likewise, common to the art, Blocks 6A and 6B represent spatial filters located at an aperture stop in order to restrict the peripheral vision of the viewer.

In order to further facilitate divergent binocular fusion the images of Blocks 1A and 1B may be made to transmit orthogonal polarizations. The two images are conveniently distinguished with pairs of sheet polarizers (e.g. cellophane sheets or poloraid sheets), a pair consisting of a polarizer and an analyzer, labeled 1Ap, 1Aa and 1Bp, 1Ba in the FIG. 1B. Within this optical layout depicted in FIG. 1C the broad spectrum optical attenuators 4A and 4B become either neutral density filters or single sheets (versus pairs of sheets) of plane polarizers.

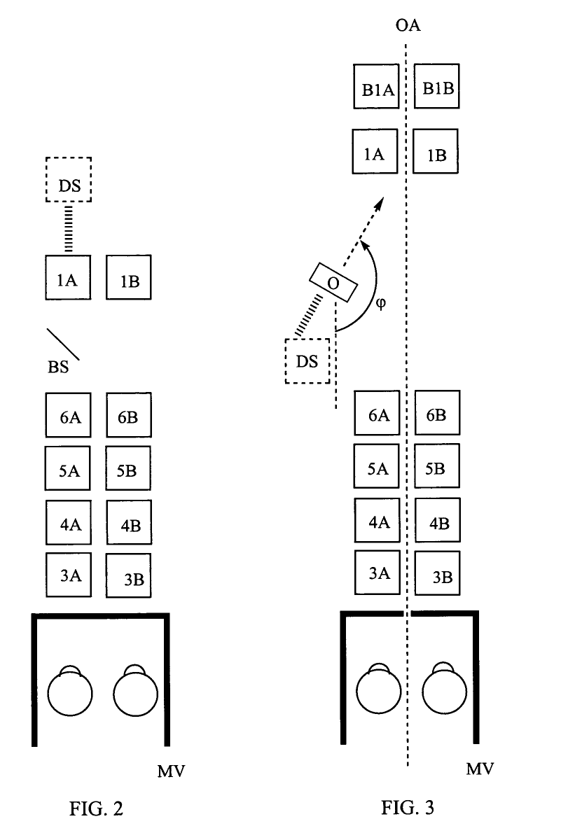

In a second embodiment of the invention the Block 1B of FIG. 1B, which represents a duplicated image, instead represents in FIG. 2 a substrate supporting a reflection of a light emitting display (i.e., an electronic color display) Block 1A from a partially transmitting surface beam splitter (labeled BS). Block 1B may be for example a mirror. The remainder of the optical layout is analogous to that of FIG. 1B. With the addition of a second display (i.e., the substrate supporting the reflection) we can physically separate the two displays to be viewed so that the distinction made by polarizing their images is no longer necessary. On the other hand, since Block 1B is a reflected image of Block 1A the control of light afforded by Block 3A must be reinstated. The stated example is not exhaustive and other possibilities may be construed by those of skill in the art.

A preferred embodiment of the invention for generating stereoscopic images under mesopic conditions is schematically outlined in FIG. 3 and is a variation of the embodiment drawn in FIG. 2. FIG. 3 shows the front surface of light emitting display Block 0 pointing at an oblique angle φ away from the viewer's optic axis OA. An image of the display is partially reflected at each of the substrates labeled Block IA and Block 1B. The substrates may be made from any number of compositions and have any value of reflectivity at any given wavelength within the visible spectrum. However, in the preferred embodiment we select transparent substrates, which simply reflect based on the contrast of refractive indices versus air. The transparent substrates represented by Blocks 1A and 1B of FIG. 3 are oriented to reflect images of the display Block 0 parallel to the viewer's optic axis OA. Note that viewing the display, as reflected by transparent substrates, is best done under dark ambient conditions, which facilitates mesopic vision. The substrates may be that of glass or of plastic where each surface of the transparent substrate reflects twice: once with the refractive index change for the light entering through the front surface and once for the light exiting from the back surface. To simplify superposition of the double images from these two surfaces the substrate should be as thin as possible. A few mm thick plastic sheet is practical. (On the other hand, if one surface is metalized, such as that of a mirror, the thickness is rather irrelevant as the reflection from the metalized surface would bury any perception of the reflection from the change of refractive indices. Analogous to eyeglasses, plastic substrates scratch more easily than glass yet are not prone to shatter upon impact).

In order to further the asymmetrical stimulation of the parvocellular and magnocellular systems of each eye over that of the previously described usage of filters, the preparation of the reflections at Blocks 1A and 1B of FIG. 3 is discussed. Each reflection may be adjusted independently in order to define subtle differences that help define the stereoscopic depth perception and other illusions upon binocular fusion. Fundamentally, one of the blocks labeled 1 must support a reflection to be made as sharp as possible in favor of preferentially stimulating the parvocellular pathway. As mentioned above the substrate must be thin enough to facilitate the superposition of the two reflected images. The transparency of the partially reflective substrate allows for the viewing of any additional media behind the substrate supporting the reflections. To further enhance luminosity contrast of a reflection at a block 1, a black absorptive medium, such as black felt, is therefore located to serve as a background. For the purpose of this embodiment, the single thin transmitting substrate is identified by Block 1A; while the black absorptive medium behind 1A is then labeled B1A. The reflected images of Block 1A would preferentially be viewed without any optical attenuation, other than perhaps a spatial filter to limit the irradiation of the perifoveal retina and/or broad spectrum attenuation in order to best stimulate the parvocellular pathway.

To continue with this embodiment, the substrate Block 1B is prepared to preferentially stimulate the magnocellular pathway. In order to compensate for the light lost upon optically filtering away that resonant with foveal cones, greater reflectivity is preferred in Block 1B versus Block 1A. This may be trivially accomplished with a glass mirror. However, in order to reduce weight, as well as the proclivity of glass to shatter, a simple superposition of plastic transparent substrates may instead be used. Thus Block 1B represents a stack of multiple reflections. Although the multitude of reflections will not be perfectly aligned when viewed with one eye, binocular fusion is found to compensate to allow a sharp resultant image. The total number of reflections that may be compensated is determined on an individual basis. The sheets comprising the stack of planar plastic substrates need only be spaced far enough apart that interference fringes from micron dimension spacing are not evident. It is emphasized that the reflected images of Block 1B would preferentially be viewed with optical attenuation of light resonant with foveal cones. In combination with these optical filters, a nonaligned multitude of reflected images further stimulates the magnocellular pathway.

Other subtleties may be incorporated for asymmetric preparation and adapted to the preferences of the individual viewer. For example, a thin transparent plastic sheet is pliable enough to induce curvature, which may be utilized to selectively distort the reflected image. Such a contrast to the other reflected image may help induce further illusions. Likewise, the background medium B1B may be chosen as either black absorptive or white scattering. Examples of background media that scatter are white paper or even an optically clear container filled with an aqueous solution of nondairy creamer. Furthermore, experimentation with color adaptation (e.g., using colored substrates in a myriad of possibilities) is viable with the present invention.

The remainder of the optical layout depicted in FIG. 3 is analogous to that of FIG. 2; identically numbered blocks represent identical functions. It is preferred to use a dual lens system for Blocks 5A and 5B. In order to view an image in the identical orientation as that of directly viewing the display, this telescopic view of reflections demands manipulation. There are a number of optical constructs that can be devised, for example, to invert upside down or to reverse a mirror image. The best solution eliminates these additional optics by a simple redesignation of the coordinate system of the display. That is, a portable display may be physically turned upside down, but often such manipulations are performed on the image at an electronic control level determined by the viewer. The stated examples are not exhaustive and other possibilities may be construed by those skilled in the art.

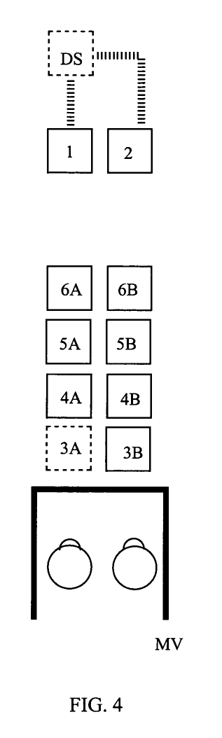

In another embodiment of the invention drawn in FIG. 4 a pair of color displays (e.g., photographs, electronic color displays) to be viewed is labeled by Blocks 1 and 2. In analogy to FIG. 1B, identically numbered blocks represent identical functions. Although the level of control may surpass the usage of optical filters to attenuate absorption by a targeted class of photoreceptor cells, this embodiment requires the extra cost of a second full color display. Furthermore, the ability of stimulating a specific neural pathway by the adjustments allowed with transparent substrates such as described with the preferred embodiment of FIG. 3 is lost. In this embodiment, we include microdisplays that are useful for generating images within a headset, e.g., as a pair of images to be viewed simultaneously by both eyes. The stated examples are not exhaustive and other possibilities may be construed by those of skill in the art.

What is claimed is:

1. A system that asymmetrically stimulates at least the parvocellular and the magnocellular pathways of the binocular visual processing of at least one set of displayed images comprising:
a means for controlling of ambient lighting conditions in order to induce mesopic vision for both eyes;
a means for storing and/or displaying at least one set of images that are either stationary or transient;
a means for blocking peripheral vision of the space outside of the said means of displaying images;
an arrangement of optics that asymmetrically stimulates at least the parvocellular and magnocellular pathways of the binocular visual processing of the at least one set of images.

2. A means as claimed in claim 1 wherein the step of controlling of ambient lighting comprises:
coarse adjustment of a room's general illumination;
fine adjustment of the light intensities reaching each eye with independent control of broad spectrum optical attenuation filters.

3. A means as claimed in claim 1 wherein the step of displaying at least one set of images is created for each eye either chemically, electronically or optically such that at least two sets of displayed images are viewed simultaneously.

4. A means as claimed in claim 1 wherein the step of creating at least one set of images for each eye comprises means for creating two sets of images, one set for each eye, which are simultaneously displayed for each eye.

5. A means as claimed in claim 1 wherein the step of blocking peripheral vision of the space outside of the said means of displaying images comprises:
spatial filters;
refractive and/or reflective and/or dispersive optics for lateral and/or angular magnification of an image.

6. A means as claimed in claim 1 wherein the step consisting of an arrangement of optics that asymmetrically stimulates at least the parvocellular and magnocellular pathways of the binocular visual processing of the at least one set of images is independently controlled with respect to each eye's view and comprises at least one of spatial and/or optical filters that are selected from the group consisting of solid, liquid or gaseous filter material.

7. A means as claimed in claim 1 wherein the step consisting of an arrangement of optics that asymmetrically stimulates at least the parvocellular and magnocellular pathways of the binocular visual processing of the at least one set of images further comprises simultaneously viewing at least two sets of images at least one set by each eye and that the at least two sets are made distinguishable in terms of size, shape, color, hue, luminosity contrast, resolution, and/or brightness.

8. A system of optics that allows independent control of at least two sets of displayed images in terms of size, shape, color, hue, luminosity contrast, resolution, and/or brightness comprising:
at least one light emitting display capable of displaying full color streaming video;
at least one substrate supporting reflections.

9. A system of optics of claim 8, wherein the display is selected from the group consisting of either the luminescent or incandescent types.

10. A system of optics of claim 8, wherein the substrate is optically homogeneous and/or supports films of differing refractive indices, material compositions and thicknesses.

11. A system of optics of claim 8, wherein the at least one substrate supporting reflections is pliable enough to distort the size and shape of the reflections and includes a plastic sheet as a means to control the size and shape of the reflections.

12. A system of optics of claim 8, wherein the at least one substrate supporting reflections is colored and is a means to affect the perception of color and hue within the images reflected on the at least one substrate.

13. A system of optics of claim 8, wherein the at least one substrate supporting reflections is optically clear to allow the viewing of additional media of color in addition to the reflection of the displayed images as a means to affect the perception of color and hue within the images reflected on the at least one substrate.

14. A system of optics of claim 8, wherein the at least one substrate supporting reflections is optically clear to allow the viewing of additional media in addition to the reflection of the displayed images in order to affect the perception of luminosity contrasts and resolution within the images reflected on the at least one substrate.

15. A system of optics of claim 14, wherein the means to affect luminosity contrasts within the images reflected on the at least one substrate further comprises:
an increase of luminosity contrasts within the images reflected on the at least one substrate based on blackbody absorption of the light transmitted through the at least one substrate by the additional media viewed through the reflected images;
a decrease of luminosity contrasts and a decrease of resolution within the images reflected on the at least one substrate based on scattering of the light transmitted through the at least one substrate by the additional media viewed through the reflected images.

16. A system of optics of claim 8, wherein the means for supporting reflections further comprises more than one substrate, not restricted in sizes, shapes, material compositions and that are separated by air.

17. A system of optics of claim 16, wherein the plurality of substrates supporting reflections further serves as a means for decreasing resolution and increasing brightness.
